(19) United States
(12) Patent Application Publication
(10) Pub. No.: US 2017/0326330 A1
(43) Pub. Date: Nov. 16, 2017

(54) MULTIMODAL PLATFORM FOR TREATING EPILEPSY

(71) Applicants: Grzegorz Bulaj, Salt Lake City, UT (US); Carol Bruggers, Salt Lake City, UT (US); Pegah Afra, Salt Lake City, UT (US); Matthew Sweney, Salt Lake City, UT (US)

(72) Inventors: Grzegorz Bulaj, Salt Lake City, UT (US); Carol Bruggers, Salt Lake City, UT (US); Pegah Afra, Salt Lake City, UT (US); Matthew Sweney, Salt Lake City, UT (US)

(21) Appl. No.: 15/594,303

(22) Filed: May 12, 2017

Related U.S. Application Data
(60) Provisional application No. 62/336,067, filed on May 13, 2016.

(51) Int. Cl. A61M 21/00; G06F 19/00; G09B 5/06; G09B 19/00

(52) U.S. Cl. CPC A61M 21/00; G09B 5/065; G09B 19/00; G06F 19/3481; A61M 2230/04; A61M 2205/502; A61M 2230/10; A61M 2021/0027; A61M 2230/63; A61M 2230/50

(57) ABSTRACT

The present disclosure describes computer systems and computer-implemented methods for treating epilepsy. An interactive user interface is configured to deliver self-care and cognitive behavioral therapy to a user, and also to deliver antiseizure music to the user, as the user engages with the interactive content. The antiseizure music includes one or more music tracks having a music profile associated with an antiseizure effect. Biometric feedback based on user response to the interactive content and/or music therapy is received, and based on the received feedback, the treatment profile is validated or is updated by adjusting the interactive content and/or music track selection.

### CROSS-REFERENCE TO RELATED APPLICATIONS

This application claims priority to and the benefit of U.S. Provisional Patent Application Ser. No. 62/336,067, filed May 13, 2016 and titled “MULTIMODAL EPILEPSY MANAGEMENT SUITE,” the disclosure of which is incorporated herein by this reference in its entirety.

### BACKGROUND

According to the World Health Organization, there are 50-60 million people living with epilepsy world-wide, and only 70% of them respond to current treatments to control their seizures. In the United States, there are estimated 2.2 million people with epilepsy, and approximately 150,000 people are diagnosed with epilepsy each year. People with epilepsy often experience affective comorbidities and lower quality of life. The diverse etiologies and complex mechanisms of epileptic seizures pose a challenge to reach long-term seizure freedom in approximately 20-30% of epilepsy patients, whereas newly diagnosed patients have only 50% chance to become seizure free after taking their first anti-seizure medication. Even after becoming seizure-free, over 60% of patients who discontinue taking anti-seizure drugs experienced relapse over a period of at least three years. Current models can predict seizure recurrence and long-term outcomes when epilepsy patients discontinue taking anti-seizure drugs. Epilepsy patients often have such comorbidities as depression and anxiety.

To improve control of seizures in patients with refractory epilepsy, non-pharmacological options range from dietary interventions (ketogenic, modified Atkins and low-glycemic diets) to neuromodulation to brain surgery. Cognitive behavioral therapy, mindfulness-based interventions, and self-management techniques may be utilized. However, these treatments are often hampered by low patient adherence and failure to stay sufficiently engaged. There is thus an ongoing need to discover and develop new therapies with improved efficacy and clinical outcomes for people with epilepsy.

### BRIEF SUMMARY

The present disclosure relates to computer systems, computer-implemented methods, and computer hardware storage devices which may be utilized for the treatment of epilepsy. Embodiments described herein provide an interactive user interface with interactive content configured to deliver self-care and cognitive behavioral therapy to a user as the user engages with the interactive content. Embodiments are also configured to provide antiseizure music to the user as the user engages with the interactive content. The antiseizure music includes one or more music tracks having a music profile associated with an antiseizure effect. The antiseizure music may be delivered as music tracks or in audiovisual form of music video. In some embodiments, feedback based on user response to the interactive content and/or music therapy is received. Based on the received feedback, a treatment profile including the interactive content and music track selection is validated or is updated by adjusting the interactive content and/or the music track selection.

Additional features and advantages will be set forth in part in the description that follows, and in part will be obvious from the description, or may be learned by practice of the embodiments disclosed herein. The objects and advantages of the embodiments disclosed herein will be realized and attained by means of the elements and combinations particularly pointed out in the appended claims. It is to be understood that both the foregoing brief summary and the following detailed description are exemplary and explanatory only and are not restrictive of the embodiments disclosed herein or as claimed.

### BRIEF DESCRIPTION OF THE DRAWINGS

In order to describe various features and concepts of the present disclosure, a more particular description of certain subject matter will be rendered by reference to specific embodiments which are illustrated in the appended drawings. Understanding that these figures depict just some example embodiments and are not to be considered to be limiting in scope, various embodiments will be described and explained with additional specificity and detail through the use of the accompanying drawings in which:

FIG. 1 illustrates a computing environment that can be used as a therapy for epilepsy and epilepsy comorbidities such as depression and anxiety;

FIGS. 2 through 7 illustrate various exemplary user interface displays showing interactive content configured to provide self-care and cognitive behavioral therapy to a user as the user engages with the interactive content;

FIG. 8 illustrates an exemplary categorization/scoring model which may be utilized to categorize/score a set of music tracks and to select music tracks having potential antiseizure activity based on the categorization/scoring; and

FIG. 9 illustrates a flowchart of an exemplary method for using an interactive user interface and antiseizure music as multimodal epilepsy treatment.

### DETAILED DESCRIPTION

### Introduction

The present disclosure relates to computer systems, computer-implemented methods, and computer hardware storage devices which may be utilized for the treatment of epilepsy. Embodiments described herein provide an interactive user interface with interactive content configured to deliver self-care and cognitive behavioral therapy to a user as the user engages with the interactive content. Embodiments are also configured to provide antiseizure music to the user as the user engages with the interactive content. The antiseizure music includes one or more music tracks having a music profile associated with an antiseizure effect. The antiseizure music is delivered as music tracks or in audiovisual form of music video. In some embodiments, feedback based on user response to the interactive content and/or music therapy is received. Based on the received feedback, a treatment profile including the interactive content and music track selection is validated or is updated by adjusting the interactive content and/or the music track selection.

The terms “digital health” and “digital therapeutics” refer to a branch of healthcare that employs digital technologies (often including mobile technologies and the internet) for improving health and/or treating specific medical conditions. Many digital health technologies (including mobile device applications) are focused on wellness and health coaching or disease self-management. Such software does not require regulatory clearance, though the United States Food and Drug Administration can and has cleared certain software for classification as a medical device.

A conventional digital health technology for epilepsy patients may be structured as a mobile device application or internet-based resource for delivering self-management content. Typically, such content is provided in the form of a “seizure diary.” Such epilepsy treatment technologies may also include content focused on managing medications, stress, and sleep.

In contrast to such conventional applications, embodiments described herein integrate music-based epilepsy therapy with interactive content. The interactive content is configured to maintain patient engagement while the music therapy is simultaneously applied. The interactive content is configured to enable self-care and cognitive behavioral therapy to the user as the user interacts with the content and while the user is exposed to the music-based therapy. The integrated, multimodal treatment systems described herein may beneficially increase patient engagement and better ensure desired exposure to music-based therapy. In some embodiments, the music-based therapy and/or interactive content treatments may be tailored to an individual patient to improve the likelihood of clinical efficacy. In some embodiments, such tailoring may occur as part of an evolving process that seeks to optimize treatment to an individual patient's present needs.

Embodiments described herein may be utilized in combination with other epilepsy treatments. For example, combination treatments may include the multimodal embodiments described herein in combination with one or more pharmaceutical compositions (i.e., antiseizure drugs), dietary treatments, neuromodulation devices, or printed paper planners having epilepsy specific content. In addition, while most of the embodiments described herein are configured to deliver antiseizure music through a computer device (such as a mobile device, laptop, or personal computer), other embodiments may additionally or alternatively deliver the antiseizure music through a peripheral device, such as an alarm clock or a children's toy.

### Computer System Overview

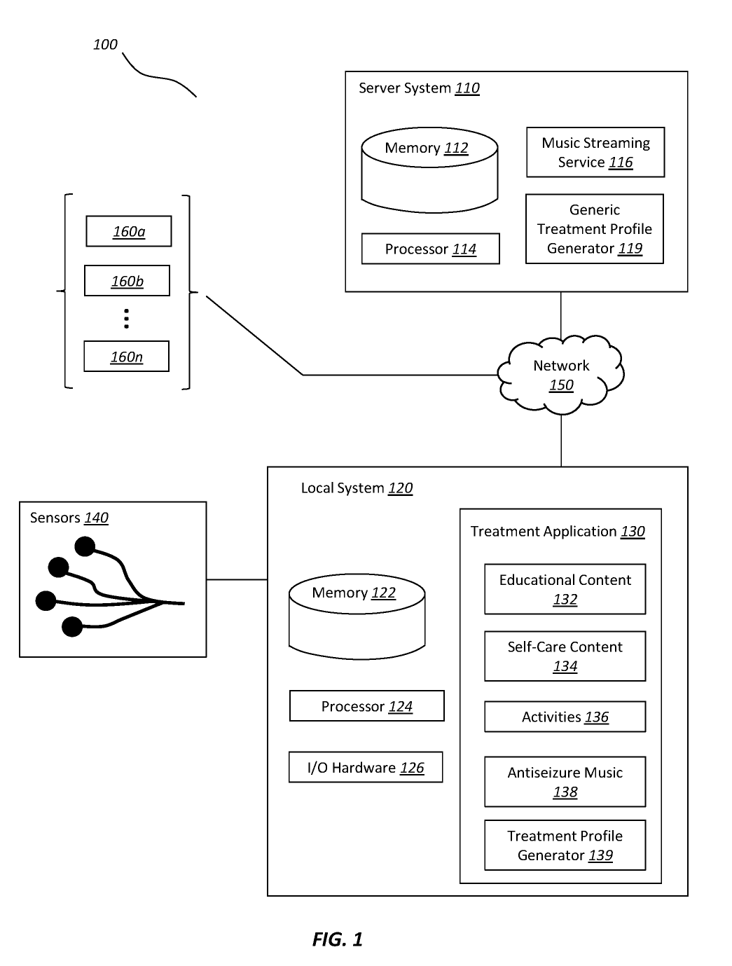

FIG. 1 illustrates an exemplary computer system configured for providing multimodal epilepsy treatment. The illustrated embodiment includes a server system 110 in communication with a local system 120. The server system 110 and local system 120 are connected by (or are part of) a network 150, such as, for example, a Local Area Network (“LAN”), a Wide Area Network (“WAN”), or the Internet. The separate computer systems may be configured to communicate with each other through suitable application programming interfaces (APIs) that enable the respective systems to communicate and share data with one another and/or with other systems (including other systems within a distributed computing environment).

As shown, the server system 110 includes memory 112 and at least one processor 114, and the local system 120 includes memory 122 and at least one processor 124. Each of memory 112 and memory 122 may independently be physical system memory, which may be volatile, non-volatile, or some combination of the two. The term “memory” may also be used herein to refer to non-volatile mass storage such as physical storage media.

As shown, the local system 120 includes input/output hardware 126. The input/output hardware may include, for example, one or more keyboards, mouse controls, touch screens, microphones, speakers, headphone connection components (wired or wireless), display screens, track balls, and the like to enable the receiving of information from a user and for displaying or otherwise communicating information to a user. In some embodiments, the local system 120 is a mobile device having a touch screen interface.

The illustrated local system 120 also includes a treatment application 130. The treatment application 130 is configured to provide to the user a variety of interactive treatment content as therapy for epilepsy. The interactive content may include, for example, educational content 132, self-care content 134, and activities 136. At least some of the interactive content is designed to engage the user and require user responsiveness and feedback as opposed to being a passive information display.

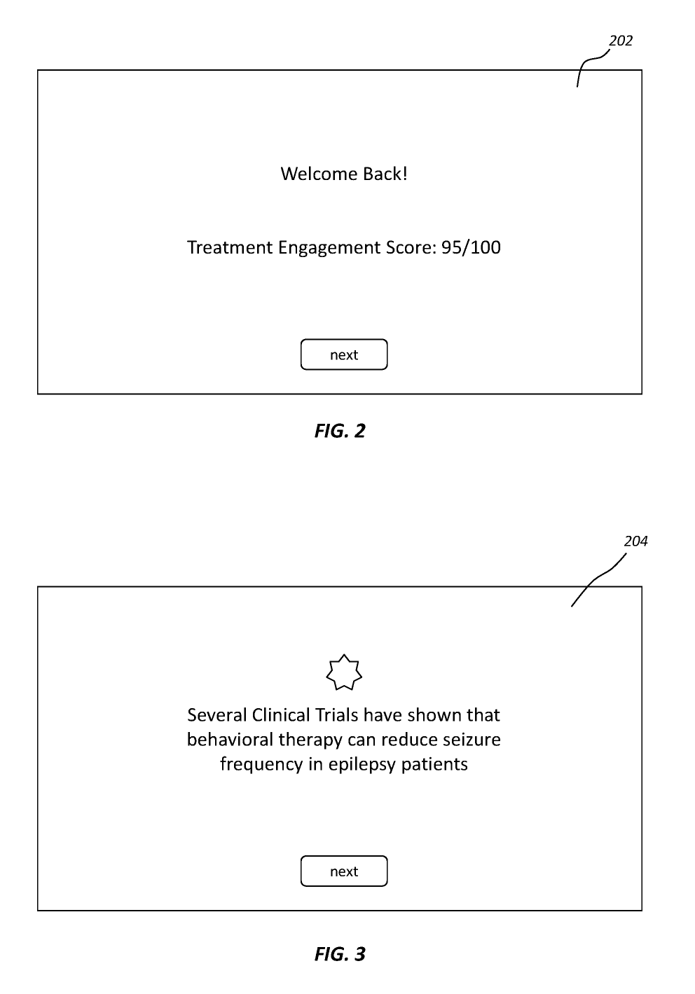

Examples of educational content 132 include epilepsy information (e.g., benefits of self-care) and epilepsy news updates (e.g., advances in clinical research). Self-care content 134 is related to self-examination and cognitive behavioral therapy. Such content is configured to prompt a patient to reflect on and record, for example, stress levels, enjoyable activities, sleep quality, relaxation level, emotions, seizure occurrences, seizure triggers, medication adherence, and gratitude. Activities 136 may include games, leisure activities, creation/art projects, and the like. The interactive content may also include a summary or “score” reflecting the level of user engagement with the application. Exemplary user interfaces for providing the interactive content are described in more detail below in relation to FIGS. 2 through 7.

As shown, the treatment application 130 also provides antiseizure music 138. Some musical selections are associated with an antiseizure effect. For example, Mozart's sonatas K.448 and K.545 have been shown to reduce seizure frequencies and epileptiform discharges in epilepsy patients. These effects have even been shown in pediatric patients with refractory epilepsy (i.e., epilepsy which is non-responsive to antiseizure medication). Other music pieces by Mozart which may be utilized an part of the antiseizure music 138 include Sonata K.448, Sonata K.545, Symphony K.551, Concerto K.482, Concerto K.207, Concerto K.218, Symphony Kv96, and Concerto K.314. Combining antiseizure music 138 with the interactive self-care and cognitive behavioral therapy content provides multimodal therapy for managing and treating epilepsy.

Embodiments described herein beneficially provide music-based therapy simultaneously with delivery of the user interface content. For example, the treatment application 130 may be configured to automatically begin playing antiseizure music once a user begins interacting with the application 130, upon signing in, upon navigating past a welcome page, etcetera. This multimodal treatment format aids in maintaining user adherence to the therapy. For example, as compared to a standalone music therapy regimen, the multimodal treatment provided by the illustrated embodiments increases the likelihood that a user will listen to the therapeutic music consistently and/or for the desired length of time.

In addition, the therapeutic music is presented under circumstances where the user is prompted to be engaged and reflective, which may enhance the effectiveness of the music therapy. For example, a patient simply instructed to listen to antiseizure music for a certain amount of time each day may be more likely to be become distracted while the music is played, to forget a treatment session, or to listen inconsistently.

The illustrated local system 120 is communicatively coupled to one or more sensors 140 configured to provide biometric information about the user. The sensors 140 may include, for example, an electroencephalogram (EEG) sensor, an electrodermal sensor, an electrocardiogram (EKG) sensor, a movement sensor (e.g., accelerometer, gyroscope), temperature sensor, heart rate monitor, and combinations thereof. The one or more sensors 140 may be provided in the form of wearable devices which a user dons during a therapy session. The local system 120 is configured to receive the biometric information gathered by the one or more sensors 140 for use in tailoring the multimodal epilepsy treatment to the particular user from which the biometric data is gathered.

The received biometric data can be used to measure the effects of the multimodal therapy in real time. For example, the delivered multimodal therapy can be considered as effective if the user's measured biometrics during therapy tend to move away from biometrics associated with risk of epileptiform activity, or if the user's measured biometrics during therapy tend to be maintained in a state inversely correlated with epileptiform activity. In contrast, the delivered therapy can be updated/adjusted if the user's biometrics trend toward biometrics associated with risk of epileptiform activity or if the user has no change from a state carrying risk of epileptiform activity.

As shown, the treatment application 130 also includes a treatment profile generator 139. The treatment profile generator 139 operates to configure a treatment profile for execution within the treatment application 130. In some embodiments, the treatment profile generator 139 configures the treatment profile for a particular user according to received responsive feedback. For example, the treatment profile generator 139 may receive responsive feedback in the form of biometric data from the one or more sensors 140 and/or in the form of the user's interaction with the delivered interactive content. In response, the treatment profile generator 139 may operate to maintain or adjust one or more parameters of the treatment profile, such as by maintaining or adjusting the types or arrangement of interactive content and/or the antiseizure music 138.

In some embodiments, the treatment profile generator 139 configures a treatment profile according to the received biometric data. For example, if the received biometric data indicates that the user is in a relaxed state or is trending toward a relaxed state, the treatment profile generator 139 may operate to maintain the current treatment profile, such as by maintaining the type of music played and/or maintaining the arrangement of interactive content. On the other hand, if the received biometric data indicates that the user is trending toward a more agitated state, the treatment profile generator 139 may operate to adjust the treatment profile, such as by adjusting the treatment music to a more calming/relaxing type and/or adjusting the arrangement of interactive content to include proportionally more content previously determined as being enjoyable to the user.

In some embodiments, the treatment profile generator 139 adjusts a treatment profile according to the user's interaction with the interactive content of the treatment application 130. For example, if a user's responses to the interactive content indicate a sub-optimal level of medication adherence, the treatment profile generator 139 may operate to increase the frequency and/or proportion of content related to medication adherence. In another example, if a user's responses to the interactive content indicate a sub-optimal level of sleep, the treatment profile generator 139 may operate to provide more educational content related to sleep. In another example, if a user's pace while working through the content is too fast, the frequency or number of activities may be increased to extend the length of the therapy session to ensure that the user is exposed to the desired duration of music therapy.

In some embodiments, the interactive content is arranged so as to take a period of time for the user to complete corresponding to a desired antiseizure music treatment time. This desired completion time period may be estimated based on the user's previous usage history, such as the user's average amount of time spent per item of interactive content, per activity, etcetera. Treatment times may vary according to particular patient needs. Suitable treatment times may range from about 5 minutes to about 30 minutes, for example, and may be recommended at a frequency of once per day, or 3 to 5 times per week, for example.

In the illustrated embodiment, the antiseizure music 138 is delivered to the treatment application 130 through streaming from a music streaming service 116 located at the server system 110. As explained in more detail below, the music streaming service 116 is configured to deliver to the treatment application 130 one or more music tracks associated with an antiseizure effect. The selection of music tracks may be made at least in part according to the individual user's history. For example, biometric data received at the local system 120 can indicate music tracks which provide a positive treatment effect to the particular user. This information can be communicated to the server system 110 so that the music streaming service 116 can select music tracks with similar musical characteristics for streaming to the treatment application 130 and/or can increase the play frequency of the effective music track. In another embodiment, the antiseizure music 138 is delivered in the audiovisual form to the treatment application 130 through video streaming from the streaming service 116 located at the server system 110.

In the illustrated embodiment, the server system 110 also includes a generic treatment profile generator 119. The generic treatment profile generator 119 is configured to obtain data generated by a plurality of different users of the treatment application. For example, different users at each of computer systems 160a through 160n may receive multimodal epilepsy therapy through the treatment application, and treatment data (e.g., biometric data and/or interaction data) from the multiple users may be received by the generic treatment profile generator 119 and used to configure a generic treatment suitable for the average user. For example, the generic treatment profile generator 119 may be utilized to determine music tracks and/or music video components generally associated with positive antiseizure effects across the population of users.

The generic treatment profile may be used as a default or baseline treatment profile for new users of the multimodal treatment application. A particular user's specific treatment profile may then be fine-tuned and more individually tailored as the particular user gains more usage history. For example, the treatment application 130 executing on the illustrated local system 120 may begin with the generic treatment profile as a baseline, and then the treatment profile generator 139 may further calibrate treatment to the individual user as the user is exposed to the music-based treatment, engages with the interactive content, and provides corresponding biometric feedback.

It will be understood that the particular computer system architecture illustrated in FIG. 1 represents only an example implementation. Other embodiments may divide the described memory, modules, components, and/or functions differently among the server system 110, local system 120, and additional computer systems 150. In some embodiments, memory components and/or program modules are distributed across a plurality of constituent computer systems in a distributed environment. In other embodiments, memory components and program modules are included in a single integrated computer system. Accordingly, the systems and methods described herein are not intended to be limited based on the particular location at which components are located and/or at which functions are performed.

### User Interface and Interactive Content

FIGS. 2 through 7 illustrate exemplary embodiments of user interfaces for displaying interactive treatment content. The user interfaces may be displayed as part of the treatment application 130 described in relation to FIG. 1, for example. As described above, the displayed content is designed to prompt active user engagement with the application as opposed to passive visualization. The interactive content thereby functions to enable the user to engage in self-care and cognitive behavioral therapy. The interactive content also functions to provide user engagement while the antiseizure music is played, increasing the likelihood that the user receives the intended exposure to the therapeutic music.

FIG. 2 shows an exemplary interface display 202 configured as a welcome screen. As shown, the interface display 202 includes an “engagement score” showing the user's level of engagement with the treatment application. The engagement score may be based on level of adherence to a prescribed treatment regimen, personal goals set by the user or healthcare provider, usage consistency, and the like. The engagement score may be represented in a variety of forms, and need not be a numerical value such as illustrated. The engagement score and/or other usage summary information may be displayed on other pages in addition to or as an alternative to display on the welcome page. For example, the score may be displayed at the end of a treatment session. In some embodiments, the score may be broken down by treatment type, including separate scores or sub-scores according to interactive content type and/or music therapy adherence, for example. The scoring feature can motivate users to stay engaged with the treatment application and provides a qualitative indication of adherence to treatment.

FIG. 3 shows an example of an interface display 204 configured to show educational content. The displayed educational content may include informative content and/or content intended to motivate the user to continue engagement and adherence to the treatment. Educational content messages intend to increase epilepsy-related knowledge and the user's self-efficacy. Educational content messages also intend to increase knowledge and the user's self-efficacy related to epilepsy comorbidities such as depression and anxiety.

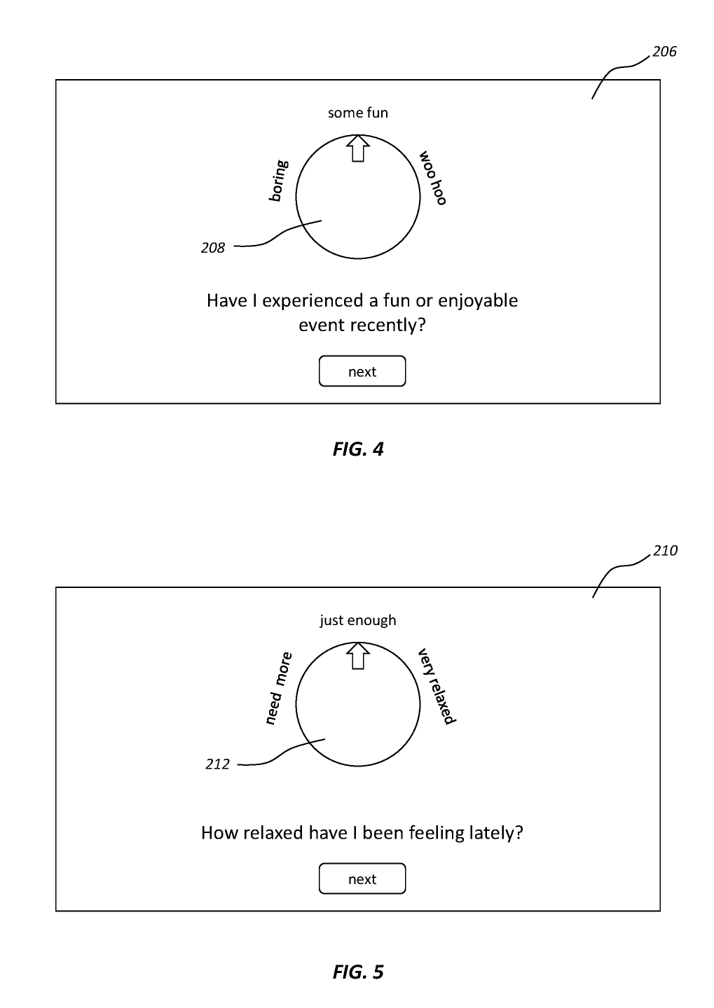

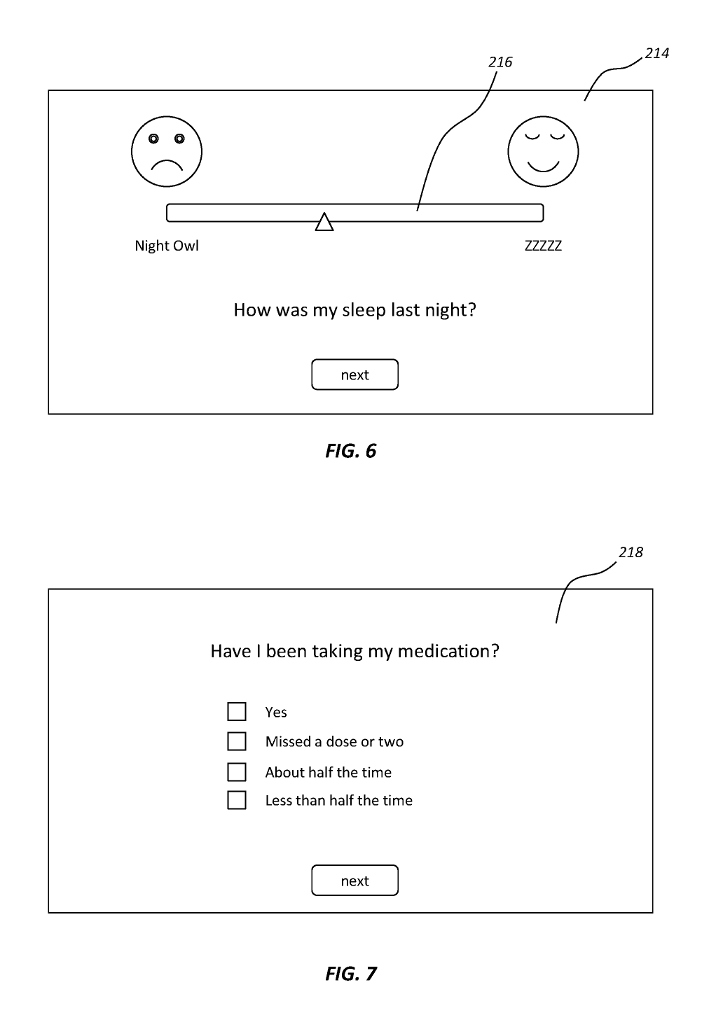

FIG. 4 illustrates an example of an interface display 206 including a user-manipulatable object 208 enabling a user to report enjoyable experience occurrences. The user-manipulatable object 208 is shown here as a digital dial. Other embodiments may additionally or alternatively include other user-manipulatable objects, such as a slider (see, e.g., FIG. 6), radio button array, checkbox option (see, e.g., FIG. 7), drop-down list, or other suitable graphical control element. FIG. 5 illustrates an example of an interface display 210 including a user-manipulatable object 212 enabling a user to report a relaxation level. FIG. 6 illustrates an example of an interface display 214 including a user-manipulatable object 216 enabling a user to report a sleep quality level. The interface displays 206, 210, and 214 are configured to prompt the user to reflect on recent experiences, relaxation levels, and sleep quality, respectively, and to thereby engage in self-care as part of the multimodal epilepsy therapy.

FIG. 7 illustrates a user interface display 218 enabling a user to report a level of medication adherence. The user interface display 218 can function as a reminder to the user.

### Antiseizure Music Selection

As described above, certain forms of music are known to be capable of providing an antiseizure effect. Mozart's sonatas K.448 and K.545 are exemplary pieces of music which may be utilized in the music-based therapy provided to the user. Embodiments described herein may also utilize other musical selections determined to be associated with an antiseizure effect. As described above, one or more music tracks may be selected based at least in part on responsive feedback from a plurality of users and/or from the particular individual user. The responsive feedback may include biometric data obtained during treatment and/or user responses to the interactive content. Particular music tracks and or particular music characteristics may be determined as positively associated with reducing epileptiform activity at a generic level based on usage data from a plurality of users and/or at an individual level based on usage data from the particular individual.

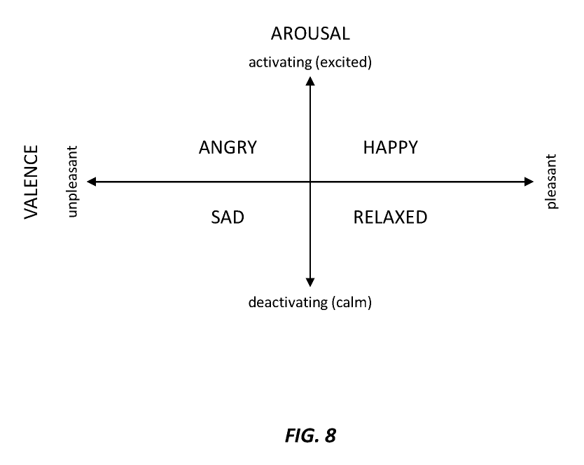

In some embodiments, antiseizure music selection is based on categorizing or scoring each music track within a larger set of music tracks, and selecting a track or set of tracks for streaming based at least in part on the categorizing/scoring. FIG. 8 illustrates an exemplary categorizing/scoring scheme based on an arousal/valence model. The arousal/valence model is one characterizing scheme that may be utilized to define a set of music tracks suitable for use in the multimodal epilepsy therapy. For example, one or more music tracks may be selected having similar categorizing/scoring within the arousal/valence model to music having known antiseizure effects, such as the Mozart pieces mentioned above. Candidate music tracks can then be validated for their physiological effects based on user responsive feedback (e.g., biometric feedback).

If the selected music track is validated as effective, the process may be iterated by then selecting one or more music tracks similar to the previously selected and now validated track. This subsequent set of candidate tracks may overlap somewhat with the previous set of candidate tracks, but the overlap may not be complete, and new potentially effective tracks may be shown as candidates. As a greater number of tracks are validated, the musical features associated with an antiseizure effect (either at the individual level or generic level) will become more apparent, and the system will be better able to apply effective antiseizure music. The foregoing iterative process may be executed, for example, by the treatment profile generator 139 and/or generic treatment profile generator 119 as described in relation to FIG. 1.

Music within the “Angry” quadrant is generally characterized as tense and furious, with loud dynamics, fast tempo, low pitches, minor key, and a relatively high number of instruments, often with an emphasis on percussion, bass, and/or drums. Music within the “Sad” quadrant is generally characterized as nostalgic and mourning, having quiet/soft dynamics, slow tempo, smooth rythm, high pitches, minor key, and relatively few instruments, often with an emphasis on strings and/or piano. Music within the “Happy” quadrant is generally characterized as carefree and triumphant, having loud dynamics, fast tempo, a mixture of high and low pitches, major key, and a relatively high number of instruments, often with an emphasis on electronic sounds, piano, and/or percussion. Music within the “Relaxed” quadrant is generally characterized as meditative and flowing, with quiet dynamics, slow tempo, smooth rythm, a mixture of high and low pitches, minor key, and relatively few instruments, often with an emphasis on acoustical instruments (e.g., acoustical guitar), strings, and/or woodwinds.

In some embodiments, music tracks may be broadly categorized according to the quadrant in which they fall. In some embodiments, music tracks may be given a two-dimensional score based on position upon the arousal/valence plane. The categorizing/scoring may be utilized to guide selection of music tracks for streaming as part of the multimodal epilepsy treatment. Patients with epilepsy often experience comorbidities of depression and/or anxiety. Accordingly, music tracks broadly falling within the Happy or Relaxed quadrants may be used as candidates for music therapy. Depending on comorbidities of epilepsy patient, the arousal/valence of the streaming content can be further adjusted. In some embodiments, patients with depression receive the streaming content with more arousal-stimulating music or music videos. In some embodiments, patients with anxiety receive the streaming content with more arousal-deactivating content. Music tracks delivered in the audiovisual form as music videos may be used to further modulate arousal/valence. In some embodiments, the streaming service may deliver music track or music videos or mindfulness-based intervention or self-care content or a combination thereof, based on individual preferences of epilepsy patients.

However, as compared non-epileptic patients having depression and/or anxiety, epileptic patients may be more sensitive to music ranking high on the arousal spectrum, as over exposure to highly activating arousal music may trigger epileptiform activity. Some embodiments therefore include limits to the allowable arousal level of streamed music. For example, a limit may be applied that limits the allowable relative jump from one music track to the next and/or within a single music track. In another example, a limit may be applied as a maximum threshold on the arousal spectrum, and only music tracks scoring below the threshold may be utilized. In some embodiments, a sliding scale is utilized. For example, the threshold for an allowable arousal score may depend on the valence score, with higher valence scores corresponding to higher allowable arousal scores and lower valence scores corresponding to lower allowable arousal scores.

### Computer-Implemented Method for Multimodal Epilepsy Treatment

The below embodiments are described with reference to acts that are performed by one or more computer systems, such as one or more of the computer systems described in relation to FIG. 1.

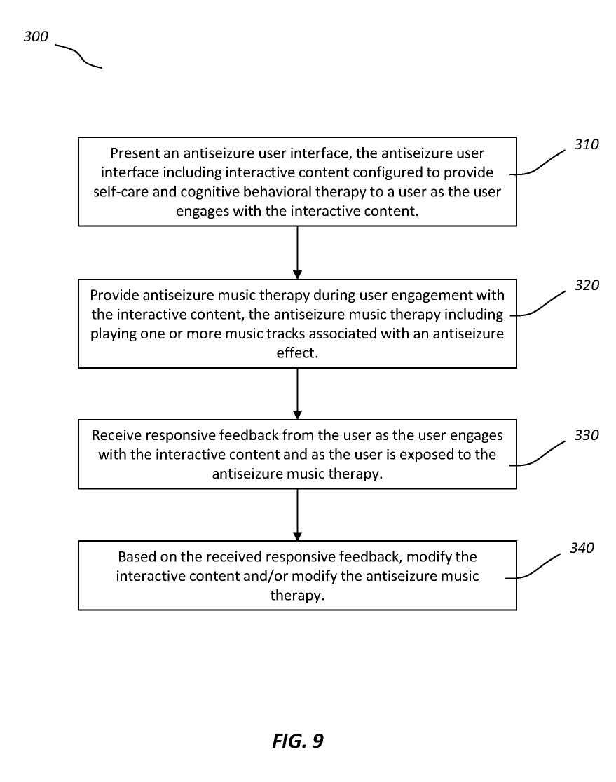

FIG. 9 is a flowchart 300 of a method for treating epilepsy using a multimodal treatment platform. A computer system presents an antiseizure user interface, the antiseizure user interface including interactive content configured to provide self-care and cognitive behavioral therapy to a user as the user engages with the interactive content (act 310). The antiseizure user interface may include one or more of the user interface displays described in relation to FIGS. 2 through 7, for example. In some embodiments, a series of interface displays providing the interactive content are provided for the user to work through during the therapy session.

The computer system also provides antiseizure music therapy during user engagement with the interactive content, the antiseizure music therapy including playing one or more music tracks associated with an antiseizure effect (act 320). The one or more tracks may be selected according to tracks known to have an antiseizure effect, according to tracks validated as providing an antiseizure effect through biometric feedback, and/or according to the process described in relation to FIG. 8.

The computer system then receives responsive feedback from the user as the user engages with the interactive content and as the user is exposed to the antiseizure music therapy (act 330). The responsive feedback may include biometric feedback and/or feedback received at the user interface as the user interacts with the interactive content. Then, based on the received responsive feedback, the computer system configures the interactive content and/or the antiseizure music therapy (act 340). For example, the computer system may modify one or more parameters of a treatment profile if the responsive feedback indicates lack of or insufficient efficacy.

### Additional Computer System Details

Embodiments of the present invention may comprise or utilize a special-purpose or general-purpose computer system that includes computer hardware, such as, for example, one or more processors and system memory. Embodiments within the scope of the present invention also include physical and other computer-readable media for carrying or storing computer-executable instructions and/or data structures. Such computer-readable media can be any available media that can be accessed by a general-purpose or special-purpose computer system. Computer-readable media that store computer-executable instructions and/or data structures are computer storage media. Computer-readable media that carry computer-executable instructions and/or data structures are transmission media. Thus, by way of example, and not limitation, embodiments of the invention can comprise at least two distinctly different kinds of computer-readable media: computer storage media and transmission media.

Computer storage media are physical storage media that store computer-executable instructions and/or data structures. Physical storage media include computer hardware, such as RAM, ROM, EEPROM, solid state drives (“SSDs”), flash memory, phase-change memory (“PCM”), optical disk storage, magnetic disk storage or other magnetic storage devices, or any other hardware storage device(s) which can be used to store program code in the form of computer-executable instructions or data structures, which can be accessed and executed by a general-purpose or special-purpose computer system to implement the disclosed functionality of the invention.

Transmission media can include a network and/or data links which can be used to carry program code in the form of computer-executable instructions or data structures, and which can be accessed by a general-purpose or special-purpose computer system. A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer system, the computer system may view the connection as transmission media. Combinations of the above should also be included within the scope of computer-readable media.

Further, upon reaching various computer system components, program code in the form of computer-executable instructions or data structures can be transferred automatically from transmission media to computer storage media (or vice versa). For example, computer-executable instructions or data structures received over a network or data link can be buffered in RAM within a network interface module (e.g., a “NIC”), and then eventually transferred to computer system RAM and/or to less volatile computer storage media at a computer system. Thus, it should be understood that computer storage media can be included in computer system components that also (or even primarily) utilize transmission media.

Computer-executable instructions comprise, for example, instructions and data which, when executed at one or more processors, cause a general-purpose computer system, special-purpose computer system, or special-purpose processing device to perform a certain function or group of functions. Computer-executable instructions may be, for example, binaries, intermediate format instructions such as assembly language, or even source code.

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, virtual or augmented reality headsets, and the like. The invention may also be practiced in distributed system environments where local and remote computer systems, which are linked (either by hardwired data links, wireless data links, or by a combination of hardwired and wireless data links) through a network, both perform tasks. As such, in a distributed system environment, a computer system may include a plurality of constituent computer systems. In a distributed system environment, program modules may be located in both local and remote memory storage devices.

Those skilled in the art will also appreciate that the invention may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources (e.g., networks, servers, storage, applications, and services). The definition of “cloud computing” is not limited to any of the other numerous advantages that can be obtained from such a model when properly deployed.

A cloud computing model can be composed of various characteristics, such as on-demand self-service, broad network access, resource pooling, rapid elasticity, measured service, and so forth. A cloud computing model may also come in the form of various service models such as, for example, Software as a Service (“SaaS”), Platform as a Service (“PaaS”), and Infrastructure as a Service (“IaaS”). The cloud computing model may also be deployed using different deployment models such as private cloud, community cloud, public cloud, hybrid cloud, and so forth.

Some embodiments, such as a cloud computing environment, may comprise a system that includes one or more hosts that are each capable of running one or more virtual machines. During operation, virtual machines emulate an operational computing system, supporting an operating system and perhaps one or more other applications as well. In some embodiments, each host includes a hypervisor that emulates virtual resources for the virtual machines using physical resources that are abstracted from view of the virtual machines. The hypervisor also provides proper isolation between the virtual machines. Thus, from the perspective of any given virtual machine, the hypervisor provides the illusion that the virtual machine is interfacing with a physical resource, even though the virtual machine only interfaces with the appearance (e.g., a virtual resource) of a physical resource. Examples of physical resources including processing capacity, memory, disk space, network bandwidth, media drives, and so forth.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A computer system configured for treating epilepsy, the computer system comprising:
one or more processors; and
one or more hardware storage devices having stored thereon computer-executable instructions which are executable by the one or more processors to cause the computer system to at least:
present an antiseizure user interface, the antiseizure user interface including interactive content configured to deliver self-care and cognitive behavioral therapy to a user as a user engages with the interactive content;
provide antiseizure music therapy during user engagement with the interactive content, the antiseizure music therapy including playing one or more music tracks associated with an antiseizure effect, the interactive content and the one or more music tracks defining a treatment profile;
receive responsive feedback from the user as the user engages with the interactive content and as the user is exposed to the antiseizure music therapy; and
based on the received responsive feedback, validate the treatment profile as effective or update the treatment profile by modifying the patient engagement content and/or the antiseizure music therapy.

2. The computer system of claim 1, wherein the interactive content includes one or more user interface displays having a user-manipulatable object, the user-manipulatable object being configured to enable the user to input a self-care metric.

3. The computer system of claim 2, wherein the self-care metric includes one or more of stress level, enjoyment, sleep quality, relaxation level, and emotions.

4. The computer system of claim 1, wherein the interactive content includes educational content related to epilepsy and/or epilepsy comorbidities.

5. The computer system of claim 1, wherein the interactive content includes a summary score reflecting a level of user engagement with the interactive content.

6. The computer system of claim 1, wherein the interactive content is arranged so as to take an estimated period of time for the user to complete, the estimated period of time corresponding to a desired antiseizure music treatment time, and the estimated period of time being based on previous usage history by the user.

7. The computer system of claim 1, wherein the antiseizure music and/or music video begins automatically upon user interaction with the interactive content.

8. The computer system of claim 1, wherein the responsive feedback includes biometric data received from one or more biometric sensors.

9. The computer system of claim 8, wherein the one or more biometric sensors include one or more of an electroencephalogram (EEG) sensor, an electrodermal sensor, an electrocardiogram (EKG) sensor, a movement sensor, temperature sensor, heart rate monitor, and combinations thereof.

10. The computer system of claim 8, wherein the treatment profile is validated in the case that the received biometric data trend away from biometric data associated with risk of epileptiform activity or in the case that the received biometric data maintain a state inversely correlated with epileptiform activity.

11. The computer system of claim 1, wherein the treatment profile is adjusted in the case that the received biometric data trends toward biometric data associated with risk of epileptiform activity or does not change from a state associated with risk of epileptiform activity.

12. The computer system of claim 1, wherein the responsive feedback includes input received at the user interface in response to user interaction with the interactive content.

13. The computer system of claim 1, wherein the one or more music tracks and/or music videos are selected at least in part based on prior responsive feedback received from the user.

14. The computer system of claim 1, wherein the one or more music tracks and/or music videos are selected at least in part based on positions of the one or more tracks upon an arousal/valence plane.

15. The computer system of claim 14, wherein the one or more music tracks and/or music videos are further selected according to a limit on allowable arousal level of the one or more music tracks.

16. The computer system of claim 1, wherein the one or more music tracks and/or music videos are selected based on being positively associated with reducing epileptiform activity according to usage data from a plurality of users.

17. A computer system configured for treating epilepsy and one or more epilepsy comorbidities, the computer system comprising:
one or more processors; and
one or more hardware storage devices having stored thereon computer-executable instructions which are executable by the one or more processors to cause the computer system to at least:
present an antiseizure user interface, the antiseizure user interface including interactive content configured to deliver self-care and cognitive behavioral therapy to a user as a user engages with the interactive content;
provide antiseizure music therapy during user engagement with the interactive content, the antiseizure music therapy including playing one or more music tracks and/or music videos associated with an antiseizure effect, the interactive content and the one or more music tracks and/or music videos defining a treatment profile for modulating user arousal;
receive responsive feedback from the user as the user engages with the interactive content and as the user is exposed to the antiseizure music therapy, the responsive feedback including biometric data received from one or more biometric sensors; and
based on the received responsive feedback, validate the treatment profile as effective or update the treatment profile by modifying the patient engagement content and/or the antiseizure music therapy,
wherein the treatment profile is validated in the case that the received biometric data trend away from biometric data associated with risk of epileptiform activity or maintain a state inversely correlated with epileptiform activity, and
wherein the treatment profile is modified in the case that the received biometric data trends toward biometric data associated with risk of epileptiform activity or does not change from a state associated with risk of epileptiform activity.

18. A computer-implemented method for treating epilepsy and/or one or more epilepsy comorbidities using a multimodal epilepsy therapy platform, the method being implemented by a computing system that includes at least one processor and one or more hardware storage devices having stored thereon computer-executable instructions that are executable by the at least one processor to cause the computing system to implement the method, the method comprising:
presenting an antiseizure user interface, the antiseizure user interface including interactive content configured to deliver self-care and cognitive behavioral therapy to a user as a user engages with the interactive content;
providing antiseizure music therapy during user engagement with the interactive content, the antiseizure music therapy including playing one or more music tracks and/or music videos associated with an antiseizure effect, the one or more music tracks and/or music videos being configured to modulate user arousal, the interactive content and the one or more music tracks and/or music videos defining a treatment profile;
provide music or music videos during user engagement to modulate the user arousal;
receiving responsive feedback from the user as the user engages with the interactive content and as the user is exposed to the music therapy; and
based on the received responsive feedback, validating the treatment profile as effective or update the treatment profile by modifying the patient engagement content and/or the music therapy.

19. The method of claim 18, wherein the interactive content includes one or more user interface displays having a user-manipulatable object, the user-manipulatable object being configured to enable the user to input a self-care metric.

20. The method of claim 18, wherein the responsive feedback includes biometric data received from one or more biometric sensors.
